(19) United States
(12) Patent Application Publication
(10) Pub. No.: US 2011/0137166 A1
(43) Pub. Date: Jun. 9, 2011

(54) TRANSDUCER ARRANGEMENT AND METHOD FOR ACQUIRING SONO-ELASTOGRAPHICAL DATA AND ULTRASONIC DATA OF A MATERIAL

(75) Inventors: Mareike Klee, Eindhoven (NL); Michael Harald Kuhn, Hamburg (DE); Karen Irene Trovato, Putnam Valley, NY (US); Christianus Martinus Van Heesch, Eindhoven (NL); Ruediger Mauczok, Eindhoven (NL); Marco De Wild, Eindhoven (NL); Biju Kumar Sreedharan Nair, Eindhoven (NL); Klaus Reimann, Eindhoven (NL)

(73) Assignee: KONINKLIJKE PHILIPS ELECTRONICS N.V., EINDHOVEN (NL)

(21) Appl. No.: 13/058,370

(22) PCT Filed: Aug. 10, 2009
(86) PCT No.: PCT/IB2009/053503

§ 371 Date: Feb. 10, 2011

Related U.S. Application Data
(60) Provisional application No. 61/089,131, filed on Aug. 15, 2008.

(51) Int. Cl. A61B 8/00

(52) U.S. Cl. 600/437

(57) ABSTRACT

The present invention relates to a transducer arrangement, particularly a transducer arrangement for acquiring tissue information, a method for using a transducer arrangement for acquiring tissue information and a glove which comprises a transducer arrangement. The transducer arrangement 21 for analysing material 40 comprises: a first transducer element 51 for inducing and receiving mechanical displacements in the material to be analysed 40; and an analysing unit 30. The transducer arrangement is arranged such as to be flexible in order to conform with a curved surface of the material to be analysed 40; and the transducer arrangement 21 is adapted to derive a first signal from a low frequency spectrum of mechanical displacements which first signal correlates to sono-elastographical properties of a material to be analysed 40; and the transducer arrangement 21 is adapted to derive a second signal from a high frequency spectrum of mechanical displacements received by the first transducer element 51 which second signal correlates to ultrasonic properties of a material to be analysed 40. With a transducer arrangement according to the invention it may be possible to generate information about the topographical anatomy and information about elastical properties of the material to be analyzed in parallel, whereby the transducer arrangement may be adapted to the unevenness of the material's surface optimally due to its flexibility which may allow the examiner or user of the transducer arrangement to analyze regions which normally may have an uneven surface profile, which may only be reached with difficulty or whose examination may cause inconvenience to the examiner as well as to the person that is being examined.

### FIELD OF THE INVENTION

The present invention relates to a transducer arrangement, particularly a transducer arrangement for acquiring tissue information, a method for using a transducer arrangement for acquiring tissue information and a glove that comprises a transducer arrangement.

### BACKGROUND OF THE INVENTION

Many forms of cancer manifest as hard lesions in soft tissue and because of this, physicians use palpation to detect presence of hard tumours within a human body.

As one example for cancer tissue detection prostate cancer is discussed here.

To screen for prostate cancer, digital rectal examination of the prostate is routinely performed on men who have reached middle age. Unfortunately, palpation is usually limited to the detection of lesions near the tissue surface and to lesions with high stiffness contrast. Even if a lesion is palpable, in general it is not possible to specify its localisation and extension exactly because digital palpation does not provide any real-time image information of the topographical anatomy in parallel. Moreover, it is usually very difficult to evaluate the lesions' quality (i.e. malignity, benignity) clearly, because this evaluation depends on the physician's subjective sensation and experience.

Two widely used medical imaging modalities, magnetic resonance imaging (MRI) and ultrasound (US) have reported accuracy levels for detecting prostate cancer, the accuracy levels being not high enough such that a significant portion of the cancerous lesions may not be detected. Studies on patients known to have prostate cancer report that one third of cancers were missed by each modality. Studies on ultrasound guided prostate biopsies found that with this technology they would have missed 20% of the men with prostate cancer. Regardless of the different accuracy levels, these imaging modalities may not provide any direct information about the elastic tissue properties.

In order to identify e.g. hard tissue associated with tumours, numerous groups are investigating ultrasound technologies. Several methods are reported, which cover compression elastography with ultrasound, transient elastography and vibration sono-elastography, making use of conventional ultrasound transducers and imaging systems.

Up to date, the examination by sono-elastography is usually done with conventional ultrasound heads.

These conventional ultrasound heads are usually rigid, i.e. inflexible, and relatively large-sized. That's why patients in general feel very uncomfortable when they are examined with such ultrasound heads intraluminally, e.g. in the rectum for detecting prostate lesions. Furthermore, some regions that should be examined are only accessible with difficulty or there is only little room in such regions that makes it difficult for the examiner to place and handle a large-sized ultrasound head correctly.

Moreover, it is necessary to provide an adequate mechanical contact, preferably wet contact, between the flexible tissue surface and the inflexible ultrasound head to guarantee a secure transmission of signals. Practically this is done by using an ultrasound gel and by pressing the ultrasound head on the tissue surface. Depending from the tissue's consistency and surface form, it is necessary to use a lot of ultrasound gel and to press the ultrasound head with high pressure on the tissue surface to reach a broad and stable contact between the tissue surface and the ultrasound head. A higher pressure on the tissue surface may cause deformation of the tissue's structure during the examination which may mask lesions of interest.

Accordingly, there may be a need for an improved transducer arrangement, particularly a transducer arrangement for acquiring tissue information and a method for using a transducer arrangement for acquiring tissue information.

### SUMMARY OF THE INVENTION

These needs may be met by the subject matter according to the independent claims. Advantageous embodiments of the present invention are described in the dependent claims.

According to a first aspect of the invention, a transducer arrangement for analysing material is proposed. The transducer arrangement comprises a first transducer element for inducing and receiving mechanical displacements in the material to be analysed; and an analysing unit. Therein, the transducer arrangement is adapted such as to be flexible in order to conform with a curved surface of the material to be analysed. Furthermore, the transducer arrangement is adapted to derive a first signal from a low frequency spectrum of mechanical displacements which first signal correlates to sono-elastographical properties of a material to be analysed and the transducer arrangement is adapted to derive a second signal from a high frequency spectrum of mechanical displacements received by the first transducer element which second signal correlates to ultrasonic properties of a material to be analysed. In other words, the first aspect of the present invention may be seen as based on the idea to e.g. provide a device which is flexible and which is adapted to detect and provide data of different properties (e.g. sono-elastographical and ultrasonic data) of a material in parallel.

The flexibility can be achieved e.g. by using one or more transducer elements which are prepared with a manufacturing technique which allows to create transducers on a flexible substrate. Additionally or alternatively, as described further below, the flexibility can be achieved by providing a multiplicity of individual transducer elements forming an entire transducer arrangement wherein the individual transducer elements are adapted such that they can be moved with respect to a respective neighbouring transducer element.

The ability of providing information on different material properties can be realised by adapting the transducer elements such that they are able to detect mechanical displacements within different frequency spectra, preferably over a wide frequency range. Knowing that the response to mechanical excitation in different frequency spectra depends on physical properties of the material to be analysed, material properties correlating to sono-elastographical properties, on the one hand, and to ultrasonic properties, on the other hand, can be derived from response signals. For example, physical properties of the material may be analysed such as elasticity, visco-elasticity and cross-link density. The mechanical excitation may be generated e.g. by the transducer element itself or manually.

With a transducer arrangement according to the first aspect of the invention it may be possible to generate e.g. information about the topographical anatomy and information about elastical properties of the material to be analyzed in parallel, whereby the transducer arrangement may be adapted to the unevenness of the material's surface optimally due to its flexibility which may allow e.g. the examiner or user of the transducer arrangement to analyse regions without applying high pressure and to analyze regions which normally may have an uneven surface profile, which may only be reached with difficulty or whose examination may cause inconvenience e.g. to the examiner as well as to the person that is being examined.

With a transducer arrangement according to the first aspect of the invention, the generation of e.g. information about the topographical anatomy of the material to be analyzed may be effected on the basis of e.g. high frequency data (e.g. ultrasonic data). Additionally, the generation of e.g. information about the elastical properties of the material to be analyzed may be effected on the basis of e.g. low frequency data (e.g. low frequency ultrasound, sound, infrasound, vibration, applying pressure manually to the material to be analyzed, etc.). Knowing these low frequency components enables a differential analysis of the tissue using the high frequency ultrasonic information.

The transducer arrangement according to the first aspect of the invention may be adapted to perform e.g. examination of the human body, e.g. of prostate, breast/mammary gland, etc. for excluding or detecting abnormalities as e.g. cancerous lesions. Further, the transducer arrangement may be adapted to perform further controlling and data processing functions, e.g. analyzing functions, displaying functions, etc.

Due to its flexibility achieved e.g. by a flexible interconnect layer between various transducer elements, the transducer arrangement may be formed in any shape, which is needed to apply it in e.g. natural orifices to realise e.g. ultrasound imaging and tissue detection with sono-elastography.

In the following, possible details, features and advantages of the transducer arrangement according to the first aspect will be explained in detail.

In the above described first aspect of the present invention, “transducer element” may be a device, e.g. electrical, electronic or electro-mechanical, that converts one type of energy or physical attribute to another for various purposes including measurement or information transfer (e.g. pressure sensors). The transducer element of the present invention may be able to send and receive data, measure and convert different attributes and transfer and/or process information related thereto simultaneously.

Each of the transducer elements may be realised in a flexible form. Further, it may be formed in various shapes, dimensions and sizes. Moreover it may be mounted with any shape so that even a 360 degree sono-elastography imaging may be possible.

“Transducer arrangement” may signify a unit which comprises an analysing unit and at least one transducer element, preferably a combination of at least two transducer elements. The transducer arrangement may comprise further components, e.g. a controlling unit, a display unit, etc.

“Analysing” may be interpreted as exploration of the material referring to different characteristics, e.g. topographical structure, elastic properties, etc. and detecting the presence and dimension of possible abnormalities compared with the physiological state or detecting pathological states as well as verifying that there are no abnormalities.

The “analysing unit” may receive analogous signals and convert them into digital signals as well as effect analysing, controlling and processing functions. The analysing unit may be separated from the transducer element or comprised in a transducer element. The analysing unit may further comprise e.g. a controlling unit, display unit, etc. The analysing unit may be coupled via cables, electrical conductors or wireless connection with at least one of the transducer elements.

“Mechanical displacements” may be interpreted as e.g. minimal movements or vibrations of the material, especially of cells or tissue. E.g. a displacement of cells and microscopical tissue structures may be evoked by ultrasonic pressure waves, a displacement of united macroscopical tissue structures may be caused by applying pressure to the material and slowly ranging the pressure e.g. manually or by inducing slow vibrations by the transducer elements.

“Material” may be e.g. all kind of tissue, including the human body, such as epithelium-tissue and endothelium tissue (e.g. surface of the skin and inner lining of digestive tract), connective tissue (e.g. blood, bone tissue), muscle tissue and nervous tissue (e.g. brain, spinal cord and peripheral nervous system).

“Inducing” may signify e.g. launching any kind of signals, e.g. ultrasound signals into or on the material and/or applying mechanical pressure into or on the material.

“Receiving” may be e.g. detecting signals (e.g. reflections, transmissions, attenuations, harmonic generation) of or from the material.

“High frequency spectrum” may be interpreted as frequencies in the range of e.g. ultrasound, which means frequencies preferably higher than 20 kHz up to 1-10 GHz.

“Low frequency spectrum” may be interpreted as frequencies lower than 20 kHz, preferably in the range of several mHz up to a few kHz. For example, if the low frequency spectrum is induced manually, the frequency range of such manual probing may be within 0.1 to 2 Hz which corresponds to a duration of mechanical excitation of 0.5 to 10 s. If the low frequency spectrum is induced by vibration of the transducer element, the frequency spectrum may range e.g. from 50 Hz to 1 kHz.

The first signal can be derived e.g. from a low frequency spectrum received by a transducer element or, alternatively, can be provided by a software, e.g. by extracting the low frequency spectrum from an analysis of the high frequency signal by digital signal processing.

Sonography, particularly medical sonography, is an ultrasound-based diagnostic imaging technique used to visualize e.g. the topographical anatomy of a variety of tissues, e.g. muscles or internal organs, their size, structures and possible pathologies or lesions without giving any direct information about the tissues' and the lesions' elastic consistency.

Elastography is based on a principle similar to manual palpation, in which the examiner detects e.g. tumours because they feel harder than surrounding tissues. In elastography, e.g. a mechanical force (compression or vibration) is applied to the e.g. soft tissues, and a conventional imaging technique such as e.g. ultrasound (US) or magnetic resonance (MR) imaging is used to create a map of soft-tissue deformation. When a discrete hard inhomogeneity, such as a tumour, is present within a region of soft tissue, a modification in the vibration amplitude will occur at its location. This forms the basis e.g. for tumour detection using elastography.

If the elastography is combined with the conventional imaging technique of ultrasound, it may be called sono-elastography. Therefore, “sono-elastographical properties” may be interpreted as a variety of properties of a material that may be detected by means of sono-elastography.

Examples for further elastography methods are compression elastography, transient elastography and vibration elastography:

In the compression elastography, compression is applied to the tissue sample, then pre-compression and post compression echo return signals are compared, using correlation techniques to calculate a strain map in the tissue.

Transient elastography uses a low frequency transient vibration to create displacements in tissue, which are then detected using pulse-echo ultrasound with conventional ultrasound transducers.

Vibration sono-elastography imaging uses real time ultrasound Doppler techniques to image the vibration pattern resulting from the propagation of low frequency (less than 1 kHz) shear waves that are propagating through deep tissue.

By means of a transducer arrangement according to the first aspect of the present invention it may be possible to obtain information of e.g. both, the topographical anatomy of a tissue and its elastic properties by one and the same transducer arrangement. Preferably, the different information can be acquired simultaneously. Therein, the transducer is realised e.g. in flexible form and, therefore, may be adjusted to the tissue's surface with high accuracy.

According to an exemplary embodiment of the present invention, the transducer arrangement further comprises at least one second transducer element, and the first and second transducer elements are arranged such as to be movable with respect to each other.

“Movable with respect to each other” may signify that one transducer element may be moved horizontally, vertically or axially or in any combination of these directions in relation to the other transducer element. In other words, the transducer elements may be displaced, rotated or distorted with respect to each other. Because of these characteristics, a transducer arrangement of two or more transducer elements may be adapted optimally to the surface of a material that should be analysed, particularly if the surface of the material is uneven.

According to an exemplary embodiment of the present invention, at least one of the transducer elements of the transducer arrangement comprise a semiconductor layer.

The “semiconductor layer” may be a layer of the transducer element which comprises e.g. semiconductor materials such as silicon and/or semiconductor components or which is a semiconductor component itself. In other words, the transducer elements may be fabricated using well established silicon technology. For example, the transducer elements may be made based on a thin silicon wafer or a silicon thin film in order to obtain sufficient flexibility. The semiconductor layer may comprise the controlling unit, the evaluation unit, the analyzing unit and/or the driving electronics. The inclusion of the semiconductor layer in the transducer elements is advantageous because it may help in significantly reducing the size of the transducer arrangement e.g. by including the control electronics directly in the semiconductor layer. The reduction of the size may in turn lead e.g. to greater patient comfort.

According to a further exemplary embodiment of the present invention, at least one of the transducer elements of the transducer arrangement comprise a piezoresistive element and/or a piezoelectric micro-machined element.

The “piezoelectric element” or “piezoresistive element” may be interpreted as a piezoelectric/piezoresistive pressure sensing or pressure generating device. On the one hand, any stress that is applied directly or indirectly to the piezoelectric element may result in a charge or voltage that may be detected by electrodes. On the other hand, by applying a voltage to the piezoelectric element, a mechanical displacement of a surface of the piezoelectric element can be provoked. Accordingly, mechanical displacements can be both, detected and generated. The piezoelectric element may be adapted to detect/generate mechanical displacements within a wide frequency range. Particularly, the piezoelectric element may be adapted to detect/generate mechanical displacements within an ultrasound frequency range of typically 1-10 MHz.

According to a further exemplary embodiment of the present invention, at least one of the transducer elements comprises a capacitive micro-machined element.

Therein, the capacitive element may be adapted to change its electric capacity value upon a pressure being applied thereto. For example, the capacitive element may have two electrodes arranged at a specific distance with respect to each other. One of the electrodes forms by itself a membrane or is attached to or embedded in a dielectric membrane layer. Upon application of pressure to the membrane, this distance of the electrodes may vary and, accordingly, the capacity induced by the spaced apart electrode will vary. Thus, mechanical displacements may be detected. Particularly, the capacitive element may be adapted to detect mechanical displacements within a low frequency range of between a few mHz and several kHz. The capacitive transducer can also be adapted to detect or generate mechanical displacements within an ultrasound frequency range of typically 1-10 MHz.

It may be advantageous to include both, piezoelectric and capacitive elements, within the same transducer arrangement. Therein, either both, the piezoelectric and capacitive element may be implemented in one or each single transducer element, or one or some of the transducer elements comprise a piezoelectric element and other transducer elements comprise a capacitive element. Therein, the piezoelectric element and the capacitive element may be adapted to operate in different frequency ranges.

Advantageously, the transducer element is adapted to receive and/or generate both, the low and the high frequency spectrum of mechanical displacements simultaneously.

According to a further exemplary embodiment of the present invention, at least one of the transducer elements comprises an piezoelectric element such as a piezoelectric layer wherein electrodes are arranged on the piezoelectric element in a side-by-side fashion on a surface of the piezoelectric element. This enables the electrodes to be formed from a single layer and, therefore, to be formed in a single formation step.

Alternatively, the electrodes may be arranged on top and bottom of the piezoelectric element.

Advantageously, a semiconductor layer is arranged in parallel with the longitudinal direction of the piezoelectric element.

In this way the deformation or changes in the shape of a whole transducer element or parts of a transducer element (e.g. membranes) may be easily detected, by using the piezoresistive effect of the semiconductor layer. The layer acts thus as a strain gauge. It can also be placed in the flexible joints between transducer elements.

According to a second aspect of the present invention, a glove comprising the transducer arrangement as described above is proposed.

The glove may be interpreted as an examination glove that comprises the transducer arrangement. The glove may be made of a variety of materials, e.g. latex. The transducer arrangement may be located on the inner surface or at the outside of the glove. Alternatively, the transducer arrangement may be incorporated into the glove material. Preferably, the transducer arrangement may be located in the region of the fingers, e.g. the index finger of the glove.

According to a further exemplary embodiment of the present invention, the glove is a disposable glove.

The glove may be produced in a low cost form. The glove may be made for single use only.

According to a third aspect of the present invention, a method for acquiring sono-elastographical data and ultrasonic data in parallel, is proposed. The method comprises the following steps: adjusting a transducer arrangement to a surface of a material to be analysed; sending a first signal into the material by the transducer arrangement, wherein the first signal induces a high frequency spectrum of mechanical displacements; receiving a second signal by the transducer arrangement based on the first signal reflected by the material, the second signal correlating to ultrasonic properties of the material to be analysed; sending a third signal into the material by the transducer arrangement, wherein the third signal induces a low frequency spectrum of mechanical displacements; receiving a forth signal by the transducer arrangement, based on a response of the material to the third signal, the forth signal correlating to sono-elastographical properties of the material to be analysed; transmitting information on the second and the forth signal to an analysing unit.

The steps of the method can be partially performed in an arbitrary order or in an order as described above. E.g. the step of sending a first signal into the material can be executed before, after or at the same time with the sending of the third signal into the material. For example, sending and detecting the high frequency signal during the application of a low frequency signal enables to monitor the displacements caused by the low frequency signal and yields information on the elastic properties of the material. Details of the procedure are given below.

For example, a first signal can be emitted before emitting the third signal. The received second signal then represents ultrasonic properties in a non-compressed state of the material to be analysed. Then, a third signal may be emitted thereby mechanically displacing or compressing the material to be analysed. From the changed second signal which is received under such compressed condition, information about the elastic properties of the material to be analysed can be derived. Therein, the first signal may be continuously emitted before and while emitting the third signal. Alternatively, the first signal may be sent before emitting the third signal and then be interrupted. Then, a third signal, e.g. in the form of a mechanical displacement/compression of the material to be analysed, may be emitted and the reaction thereto may be derived from again emitting the first signal and analysing the changed second signal.

The transducer arrangement used in the method may be the transducer arrangement as described above with respect to the first aspect.

The transducer arrangement may be adapted to the surface of the material that should be analysed. In general, the surface of such materials is not planar. It is necessary to reach a continuous contact between the surface of the material and the transducer arrangement to get an optimal connection of the signals that are sent to and received from the material. Because of the flexible layout of the transducer arrangement it may be possible to get an optimal adjustment between the transducer arrangement and the material, even if the surface of the material is very uneven.

In a further step, a high frequency signal (first signal), e.g. ultrasound, may be transmitted from the transducer arrangement into the material that should be analysed. This signal may be reflected in the material depending from the material's specific structural properties, e.g. topographical anatomy of a tissue. The resulting signal (second signal), representing the reflected high frequency signal, may be transmitted from the material to the transducer arrangement and may be received by the transducer arrangement. This resulting signal comprises the information from which the structure of the material, e.g. the topographical anatomy of the tissue, may be obtained in a possible subsequent analysing step.

In a further step, a low frequency signal (third signal), e.g. vibration, may be transmitted from the transducer arrangement into or on the material that should be analysed. The high frequency signals transmitted and received under the compressed state give information on the elastic properties of a tissue. The low frequency signal itself might also be received or monitored by the transducer arrangement by pressure detectors as described above. This step is not needed if the magnitude, phase and lateral distribution of the low frequency signal is known from the properties of the actuator that emits the low frequency signal. In that case, the “third signal” would be the actuation signal.

The low frequency signal can also be derived from an analysis of the high frequency signal, if the high frequency signal is periodically applied and monitored. This can for example be used in case the low frequency signal is generated manually and/or no low frequency detectors are implemented in the array.

In a further step, signals, e.g. the second and fourth signal, may be transmitted to an analyzing unit. This analyzing unit may process the received signals so that they may be visualized e.g. at a display which may e.g. be a part of the analyzing unit.

The adjustment to the material's surface, the sending and/or the receiving of the high frequency signal and the low frequency signal and/or the transmission of the information to the analysing unit may take place simultaneously.

According to a further exemplary embodiment of the present invention, the step of transmitting information to the analysing unit also comprises transmitting the third signal.

The third signal may be needed by the analyzing unit for the further processing, e.g. if the third signal is triggered manually. E.g. when the physician manually applies pressure to the material, which induces a low frequency spectrum of mechanical displacements, a further ultrasound signal can be transmitted into the material under the pressure conditions and a forth signal, which corresponds to the reflected ultrasound signal under the pressure conditions can be received.

According to a further exemplary embodiment of the present invention, the steps of sending a high frequency signal and detecting a low frequency signal are effected by the same transducer.

It has to be noted that embodiments of the invention are described with reference to different subject matters. In particular, some embodiments are described with reference to method type claims whereas other embodiments are described with reference to apparatus type claims. However, a person skilled in the art will gather from the above and the following description that, unless other notified, in addition to any combination of features belonging to one type of subject matter also any combination between features relating to different subject matters is considered to be disclosed with this application.

The aspects defined above and further aspects, features and advantages of the present invention can also be derived from the examples of embodiments to be described hereinafter and are explained with reference to examples of embodiments. The invention will be described in more detail hereinafter with reference to examples of embodiments but to which the invention is not limited.

### BRIEF DESCRIPTION OF THE DRAWINGS

FIG. 1 shows a schematic representation of an array of transducer elements according to an embodiment of the present invention where the piezoelectric layer is actuated by an electric field in the plane of the layer, operating in the so-called d33 mode.

FIG. 2 shows a schematic representation of a transducer element according to another embodiment of the present invention where the piezoelectric layer is actuated by an electrical field perpendicular to the piezoelectric plane, operating in the so-called d31 mode.

FIG. 3 shows a schematic representation of a transducer element including an integrated capacitive pressure sensor according to another embodiment of the present invention.

FIG. 4 shows a schematic representation of a transducer arrangement according to an embodiment of the present invention which fits closely to an uneven material surface comprising a lesion that has a higher consistency than the surrounding material, wherein no pressure is applied to the material's surface.

FIG. 5 shows a schematic representation of a transducer arrangement according to an embodiment of the present invention which fits closely to an uneven material surface comprising a lesion that has a higher consistency than the surrounding material, wherein pressure is applied to the material's surface.

FIG. 6 shows a schematic representation of the signalling pathways of the signals between a transducer element and the material to be analysed and vice versa according to an embodiment of the invention.

FIG. 7 shows a schematic representation of an examination glove that comprises a transducer arrangement according to an embodiment of the present invention.

The illustration in the drawings is schematically only and not to scale. It is noted in different figures, similar elements are provided with the same reference signs.

### DETAILED DESCRIPTION OF PREFERRED EMBODIMENTS

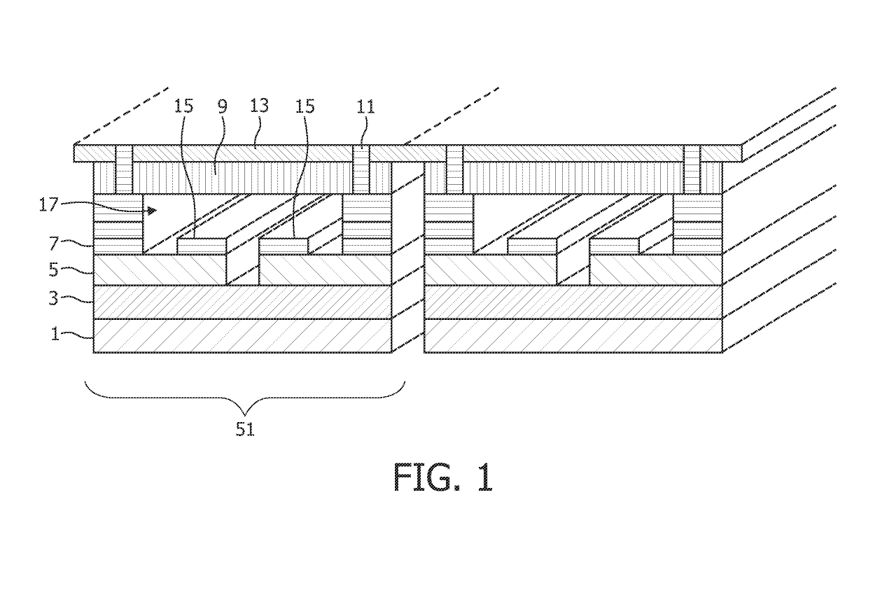

In FIG. 1 a flexible thin film ultrasound transducer arrangement according to an embodiment of the present invention is shown schematically.

It is a thin film flexible ultrasound transducer arrangement operating in the d33 mode.

In the d33 mode, also called longitudinal mode, the elongation of the piezoelectric layer is arranged in parallel to the direction of the applied voltage.

The figure shows two transducer elements 51, but the principle may be extended to 1D as well as 2D arrangements with numerous elements.

The piezoelectric transducer includes a membrane 1 and 3 formed on a substrate which is removed after formation of the transducer to allow movement of the membrane. The membrane is an inorganic material for example be composed of silicon nitride (e.g. membrane 1) and silicon oxide (e.g. membrane 3). Also a stack comprising the inorganic membrane and a barrier layer such as titanium oxide or aluminium oxide or zirconium oxide can be applied. Piezoelectric material 5, which may be lead titanate zirconate which is either undoped or doped with e.g. lanthanum (La) or any other suitable piezoelectric material, is formed on the membrane 1, 3 which, for example, may be patterned if desired to increase performance. Further, a pair of electrodes 7 and 15, which comprises for example a stack of titanium and gold or any other suitable electrically conductive material, is formed as a layer over respective regions of the patterned piezoelectric material.

When a positive voltage is applied to the inner edge electrodes 15 and a negative voltage is applied to the outer edge electrode 7, which may alternatively be grounded, elongation of the piezoelectric layers results in a downward bending. Reversing the polarity of the voltages applied to the electrode pairs, bends the membrane stack upward. Voltage pulses or any alternating current (AC) signal applied to the piezoelectric layers creates ultrasonic waves.

On top of these elements a thin film substrate 9 is mounted along the metal pads 7 using e.g. ultrasonic bonding. But also any other bonding technique, such as thermal compression, can be applied. The substrate can be for example a thinned down silicon (Si) substrate with or without integrated electronics as well as with or without an isolation layer. But also any other substrate can be mounted. In the silicon substrate, isolated vias with metal interconnects 11 are realised. Along these interconnects the elements are connected using a flexible foil 13, which comprises multi-level interconnects for signal and ground connection. To realise a flexible device, the membranes between the various elements are separated.

The driving electronics are either implemented in the thin film substrate 9, which is mounted on top of the membrane or is applied in a separate chip. To make the device ready for the application a biocompatible protection layer e.g. from parylene or any other organic or inorganic coating is applied (not shown in FIG. 1).

Due to the flexible interconnect layer between the various elements, the arrangement can be formed in any shape, which is needed to apply it in natural orifices to realise ultrasound imaging and tissue detection with sono-elastography.

In an embodiment of the invention, the flexible device shown here, does not only enable sono-elastography measurements but also can comprise pressure sensors, which enables the physician to obtain with this device more quantified data on the tissue hardness compared with the digital rectal examination. The pressure sensors integrated in the transducers can in one part of this invention be built out of piezoelectric pressure sensors.

Here the stress applied to the piezoelectric element results in electrical charge that can be detected on the electrodes.

This is one way to enable a force feedback for the physician, so that he is able detect the tissue hardness and do a sono-elastography image with the same device.

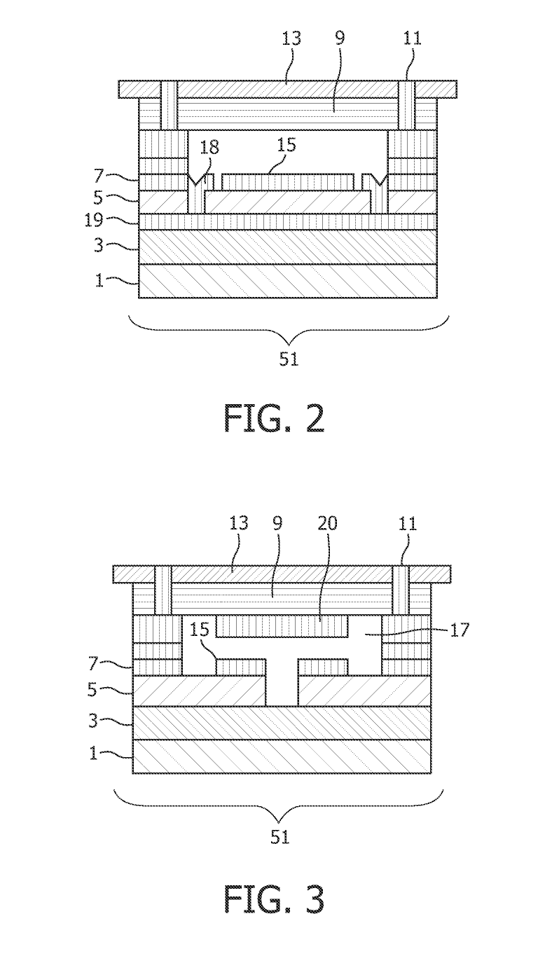

FIG. 2 shows a schematic representation of a transducer element where the piezoelectric layer is actuated by an electrical field perpendicular to the plane of the piezoelectric layer 5. Here, electrodes are mounted on the top side 15 and the bottom side 19 of the piezoelectric layer 5, sandwiching the piezoelectric material. Applying a voltage pulse, results in an elongation of the piezoelectric layer in field direction and in a contraction of the piezoelectric layer perpendicular to the electrical field, thus in the field plane. This gives rise to a bending of the membrane and ultrasound waves are transmitted.

In FIG. 3 a flexible thin film ultrasound transducer element including an integrated capacitive pressure sensor according to another embodiment of the present invention is shown schematically. Here, a conductive layer serves as one electrode 20 of the capacitive sensor element and the electrodes 15 as the second electrode of the capacitive sensor element. The conductive layer can be a highly doped Si layer that is isolated by a SiO₂ layer from the substrate 9. Alternatively, the electrode 20 can be any metal layer formed on e.g. a Si substrate with an isolation layer, or the thin film substrate 9, which can be for example bonded Si, contains a locally deposited metal electrode or forms itself an electrode 20. The two electrodes 15 on top of the piezoelectric layer 5 in the center of the device serve as the other electrodes of the capacitor with a gas or vacuum dielectric 17. Stress applied to the membranes 1,3 results in a membrane deformation and in a change of the capacitance, which can be detected.

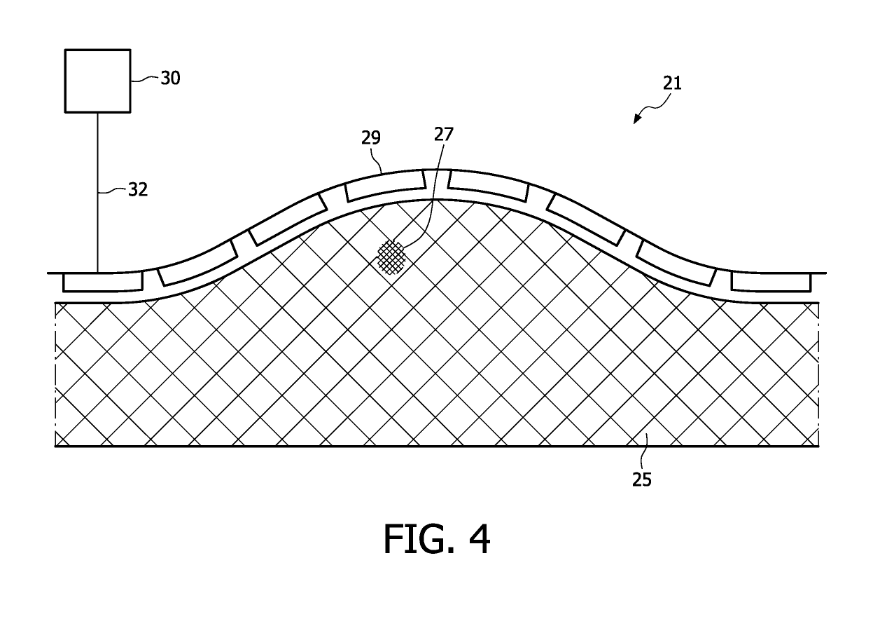

In FIG. 4 a transducer arrangement 21 according to an embodiment of the present invention that fits closely to an uneven material surface is shown schematically. Because of its flexibility the transducer arrangement may fit closely to the unevenness of a surface. The material comprises a lesion 27 that has a higher stiffness than the surrounding tissue material. The whole material 25 is uncompressed because no pressure is applied to the material's surface. The lesion does not cause any relevant change of the surface relief. Hence, no relevant stress is applied to the membranes of the transducer element 29 that is located on the surface of the lesion's region.

As shown in FIG. 4, the transducer elements can be connected to an analyzing unit 30 which is externally arranged from the transducer elements 29. The analysing unit 30 can be coupled via cables 32 or electrical conductors or wireless connection with at least one of the transducer elements. Alternatively, the analyzing unit or a part of the analyzing unit can be comprised in at least one of the transducer elements 29.

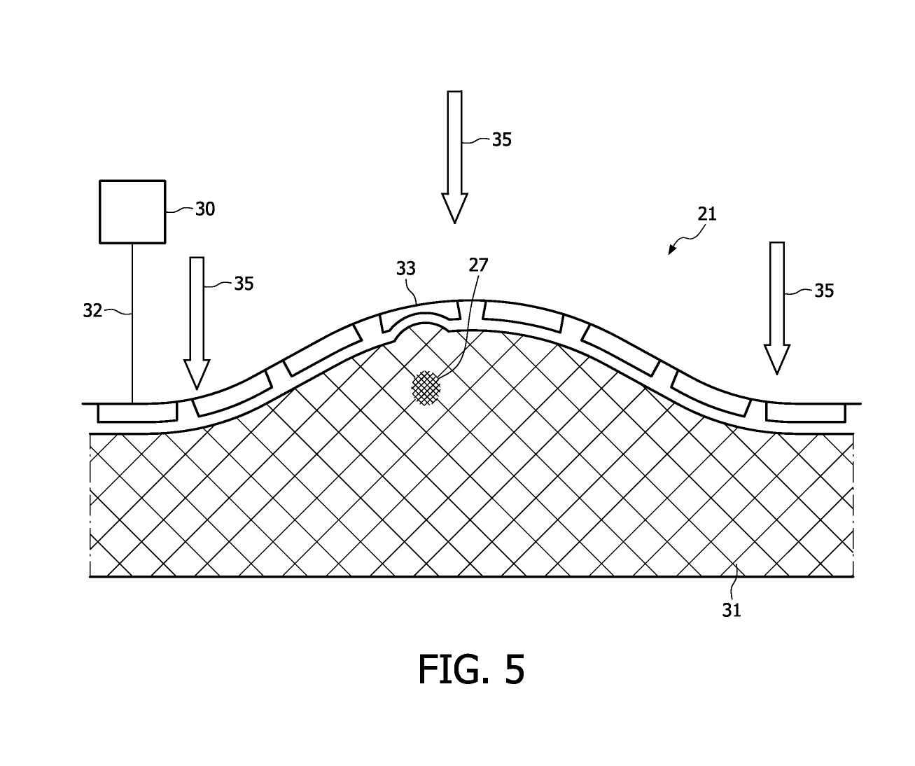

In FIG. 5 a transducer arrangement 21 according to an embodiment of the present invention which fits closely to an uneven material surface comprising a lesion 27 that has a higher consistency than the surrounding material, wherein pressure 35 is applied to the material's surface, is shown schematically. Because of a pressure applied to the material (e.g. by pressing the transducer element on the material's surface) the whole material is compressed 31. The material's regions that do not comprise any lesions of higher consistency, are compressed stronger than a material's region that comprises a lesion 27 that has a higher stiffness than the surrounding material. This causes a change of the surface relief (e.g. protrusion) or a change of the material's resistance in the region that comprises a lesion 27. This results in a rise of stress that affects to the membrane of the transducer element 33 that is located on the surface of the lesion's region. The stress applied to the membrane results in a deformation of the membrane and in a consecutive charge and/or change of the capacitance that can be detected on the electrodes.

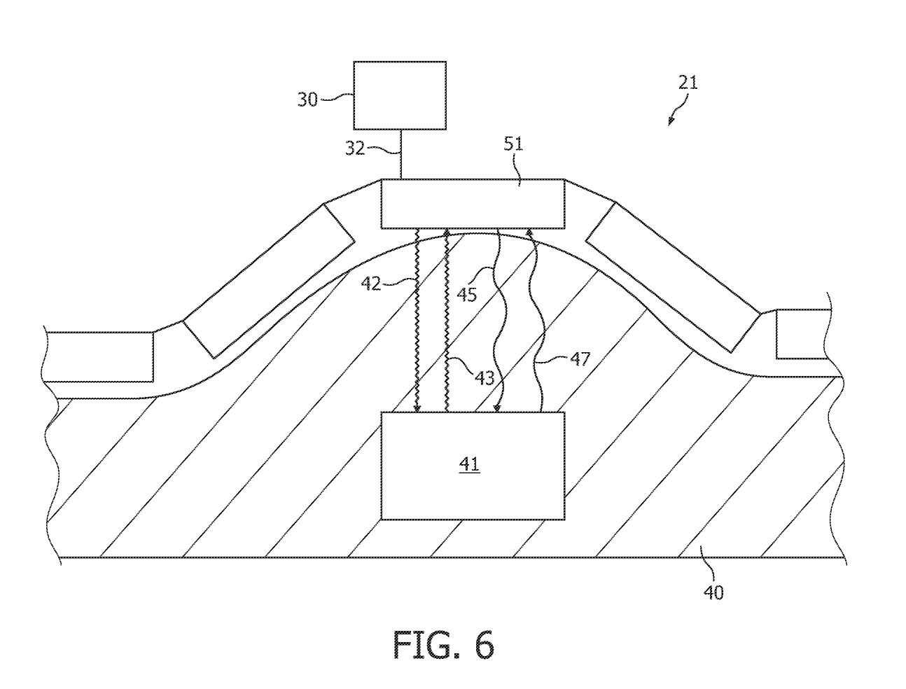

In FIG. 6 the signalling pathways of the signals between a transducer element 51 and the material to be analysed and vice versa according to an embodiment of the invention are shown schematically.

One special region 41, which represents a part of the whole material that is being analysed by the transducer arrangement, is selected to illustrate the different signalling pathways schematically.

The first signal 42 can represent a high frequency signal, e.g. ultrasound, that is transmitted from the transducer element into the material. This signal can be reflected at boundaries of the material depending on the material's specific structural properties. Hence, the resulting signal/second signal 43 represents the reflected high frequency signal and comprises information about the architecture of the material. This second signal can be transmitted from the material to the transducer element 51 and can be received by the transducer element 51. This signal can be further processed in the analyzing unit 30.

The third signal 45 can represent a low frequency signal, e.g. vibration or, alternatively, pressure which can be applied manually to the material's surface by the examiner, that is transmitted from the transducer arrangement into or on the material. This signal may be reflected in or on the material depending from the material's specific elastic properties. At very low frequencies the transmitted and reflected signals overlap each other, and it is enough to record the quasistatic pressure with element 51 to get an impression of the pressure signal in the tissue of interest 41. High and low frequency signals can be recorded simultaneously.

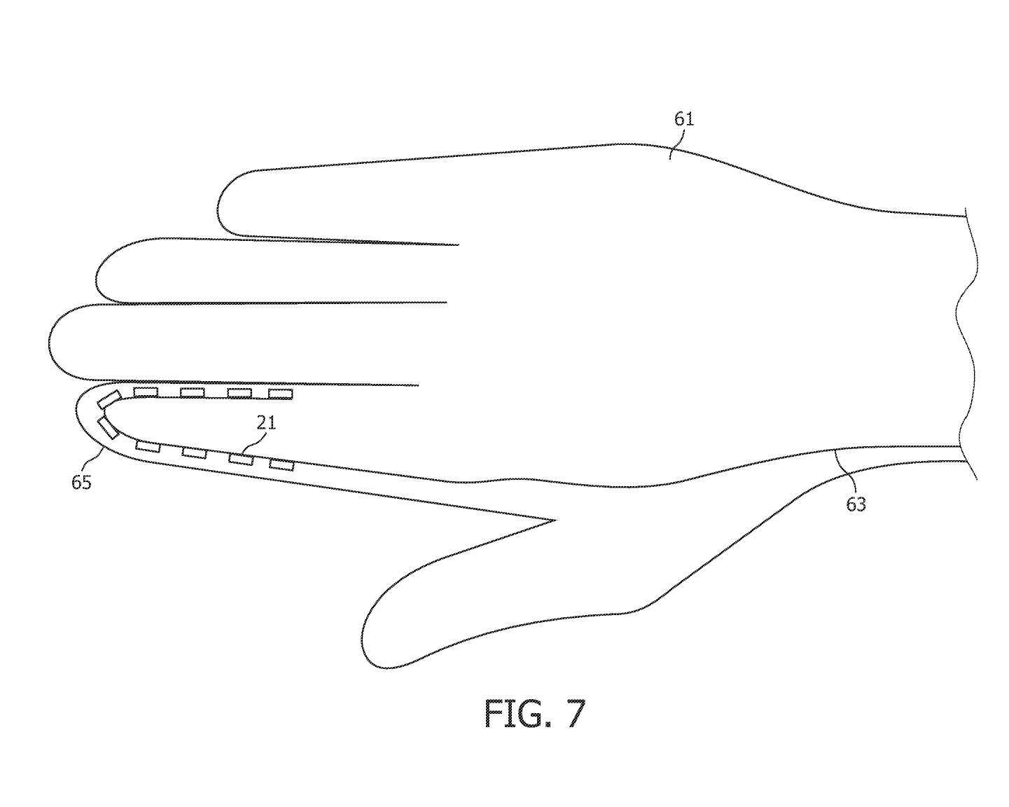

In FIG. 7 an examination glove 61 which comprises a transducer arrangement 21 according to an embodiment of the present invention is shown schematically. Preferably, the transducer arrangement is located in the palmar region of the center of the index finger up to the finger tip 65 of the glove. Alternatively, the transducer arrangement can be located at any region of the glove or various transducer arrangements at various regions of the glove can be used. The transducer arrangement can be located at the inner or outer surface of the glove, or it can be incorporated inside the glove material. The transducer arrangement can formed out as a linear array, but also as a 2D array or any other suitable form. Data transmission to and from the transducer arrangement is effected by a cable 63, alternatively by electrical conductors or wireless connection.

It should be noted that the term “comprising” does not exclude other elements or steps and the “a” or “an” does not exclude a plurality. Also elements described in association with different embodiments may be combined. It should also be noted that reference signs in the claims should not be construed as limiting the scope of the claims.

### LIST OF REFERENCE SIGNS

1 membrane
3 membrane
5 piezoelectric layer
7 outer edge electrode
9 substrate
10 flexible interconnect layer
11 isolated vias
13 flexible foil
15 inner edge/top side/center electrode
17 cavity
18 connection to bottom electrode
19 bottom electrode
20 capacitor electrode
21 transducer arrangement
25 uncompressed material
27 lesion
29 transducer element on the surface of the lesion's region, whereby the element's membranes are not affected by relevant stress
30 analyzing unit
31 compressed material
32 cable
33 transducer element on the surface of the lesion's region, whereby the element's membranes are affected by stress
35 pressure applied to the material's surface
40 material
41 special region of material that is analysed
42 first signal
43 second signal
45 third signal
47 fourth signal
51 transducer element
61 examination glove
63 cable for data transmission
65 index finger

1. Transducer arrangement (21) for analysing material (40) comprising:
a first transducer element (51) for inducing and receiving mechanical displacements in the material to be analysed (40); and
an analysing unit (30);
wherein the arrangement is arranged such as to be flexible in order to conform with a curved surface of the material to be analysed (40);
wherein the transducer arrangement (21) is adapted to derive a first signal from a low frequency spectrum of mechanical displacements which first signal correlates to sono-elastographical properties of a material to be analysed (40) and
wherein the transducer arrangement (21) is adapted to derive a second signal from a high frequency spectrum of mechanical displacements received by the first transducer element (51) which second signal correlates to ultrasonic properties of a material to be analysed (40).

2. Transducer arrangement according to claim 1, further comprising
a second transducer element for inducing and receiving mechanical displacements in the material to be analysed (40);
wherein the first and second transducer elements (51) are arranged such as to be movable with respect to each other;
wherein the transducer arrangement (21) is adapted to derive a first signal from a low frequency spectrum of mechanical displacements received by at least one of the first and second transducer elements (51) which first signal correlates to sono-elastographical properties of a material to be analysed (40); and
wherein the transducer arrangement (21) is adapted to derive a second signal from a high frequency spectrum of mechanical displacements received by at least one of the first and second transducer elements (51) which second signal correlates to ultrasonic properties of a material to be analysed (40).

3. Transducer arrangement according to claim 1,
wherein the first transducer elements comprises a semiconductor layer.

4. Transducer arrangement according to claim 1,
wherein the first transducer element (51) comprises at least one piezoresistive element, a piezoelectric micro-machined element (5) and/or a capacitive micro-machined element.

5. Transducer arrangement according to claim 4,
wherein the capacitive micro-machined element is adapted to receive the low frequency spectrum of mechanical displacements.

6. Transducer arrangement according to claim 1,
wherein the first transducer element (51) is adapted to receive both, the low and the high frequency spectrum of mechanical displacements.

7. Transducer arrangement according to claim 1,
wherein the low and the high frequency spectrum of mechanical displacements are received by different transducer elements, respectively.

8. Transducer arrangement according to claim 1,
wherein the first transducer element (51) comprises a piezoelectric layer (5); and
wherein electrodes are arranged on the piezoelectric element (5) in a side-by-side fashion on a surface of the piezoelectric element.

9. Transducer arrangement according to claim 1,
wherein the first transducer element (51) comprises a piezoelectric layer (5); and
wherein electrodes are arranged on top (15) and bottom (19) of the piezoelectric layer (5).

10. Glove (61) comprising the transducer arrangement according to claim 1.

11. Glove according to claim 10,
wherein the glove (61) is a disposable glove.

12. Method for acquiring sono-elastographical data and ultrasonic data of a material (40) in parallel comprising the following steps:
adjusting a transducer arrangement (21) to a surface of a material to be analysed (40);
sending a first signal (42) into the material by the transducer arrangement (21),
wherein the first signal (42) induces a high frequency spectrum of mechanical displacements;
receiving a second signal (43) by the transducer arrangement (21) based on the first signal reflected by the material, the second signal (43) correlating to ultrasonic properties of the material to be analysed (40);
sending a third signal (45) into the material using the transducer arrangement (21),
wherein the third signal (45) induces a low frequency spectrum of mechanical displacements;
receiving a forth signal (47) based on a response of the material to the third signal (45), the forth signal (47) correlating to sono-elastographical properties of the material to be analysed (40);
transmitting information on the second (43) and the forth (47) signal to an analysing unit (30).

13. Method for acquiring sono-elastographical data and ultrasonic data in parallel according to claim 12,
wherein the step of transmitting information to the analysing unit (30) also comprises transmitting the third signal (45).

14. Method for acquiring sono-elastographical data and ultrasonic data in parallel according to claim 12,
wherein the steps of sending the first signal (42) and receiving the fourth (47) signal are both effected by the transducer arrangement (21).

15. Method for acquiring sono-elastographical data and ultrasonic data in parallel according to claim 12.
